(19) United States
(12) Patent Application Publication
(10) Pub. No.: US 2026/0108563 A1
(43) Pub. Date: Apr. 23, 2026

(54) COMPOSITION FOR IMPROVING OR MAINTAINING MUSCLE QUANTITY OR MUSCLE STRENGTH

(71) Applicant: NIHON PHARMACEUTICAL CO., LTD., Tokyo (JP)

(72) Inventor: Tatsuya WADA, Tokyo (JP)

(73) Assignee: NIHON PHARMACEUTICAL CO., LTD., Tokyo (JP)

(21) Appl. No.: 19/100,791

(22) PCT Filed: Aug. 3, 2023
(86) PCT No.: PCT/JP2023/028458

§ 371 Date: Dec. 29, 2025

(30) Foreign Application Priority Data
Aug. 4, 2022 (JP) 2022-124661

(51) Int. Cl. A61K 35/60; A61K 9/20; A61P 21/00

(52) U.S. Cl. CPC A61K 35/60; A61K 9/2009; A61K 9/2013; A61K 9/2054; A61P 21/00

(57) ABSTRACT

The present invention provides a novel method that can improve or maintain muscle quantity or muscle strength, and a novel method that can prevent obesity, or lower body fat percentage, or lower body fat quantity, or lower fat removal quantity. The proteoglycan used in the present invention is obtained by a method for producing the proteoglycan, comprising the steps of immersing a biological sample comprising the proteoglycan in an aqueous solution of a surfactant and collecting the solution after the immersion.

### TECHNICAL FIELD

The present invention relates to a composition and a method for improving or maintaining a muscle quantity or muscle strength as well as a composition and a method for preventing obesity, or lowering body fat percentage, or lowering body fat quantity, or lowering fat removal quantity, each comprising specific proteoglycans.

### BACKGROUND ART

In Japan, the average life expectancy and healthy life expectancy are discrepant by about 10 years, and the extension of healthy life expectancy is an urgent issue. Of the major causes of the need for care, the decline in musculoskeletal systems, such as joint diseases, weakness due to old age, and fractures and falls, account for about 35% of the total, and about 50% when limited to those who require assistance. In other words, to extend healthy life expectancy, it is considered to be important to maintain healthy musculoskeletal systems (skeletal muscles, joints, and bones). However, it is well known that muscle quantity decreases with age, and it has been known that in addition to aging, inflammation and a decrease in activities of daily living (ADL) associated with knee discomfort and pain have been shown to cause a decrease in muscle quantity and muscle strength.

Proteoglycan is a general term for glycoproteins that have extremely complex and diverse structures in which several to several dozen linear sugar chains covalently bond to a single core protein, and chondroitin sulfate is a typical example of a sugar chain contained in proteoglycans existing in cartilage tissue.

Chondroitin sulfate is an ingredient that is attracting industrial attention due to its high usefulness, such as excellent moisture retention, biocompatibility, and lubricity, and various methods have been developed to effectively collect and produce it from natural resources.

In cartilage tissue, chondroitin sulfate does not exist by itself but exists in the form of a complex formed through covalent bonding with proteins, namely in the form of proteoglycans. However, it is often difficult to extract proteoglycans as they are due to their complex structure as glycoprotein complexes, and therefore, methods have been mainly employed to extract only chondroitin sulfate by thoroughly degrading the core protein portion of proteoglycans. The products of this method are mucopolysaccharides such as chondroitin sulfate.

On the other hand, there have been attempts to collect, produce, and utilize proteoglycans as they are, rather than as chondroitin sulfate. In particular, cartilage tissues of fishes, birds, and mammals contain proteoglycans with chondroitin sulfate as the main sugar chain, while these cartilage tissues are usually disposed of as waste, so that several methods for the production of proteoglycans from cartilage tissues have been proposed to make effective use of the waste as well.

For example, Patent Document 1 discloses as a method for producing proteoglycans from cartilage tissue, a method for producing proteoglycans, comprising immersing a biological sample such as cartilage tissue comprising proteoglycans in an aqueous solution of a surfactant and collecting the solution after the immersion. And, the proteoglycans disclosed in Patent Document 1 are known to be useful for the prevention, reduction, or treatment of symptoms of arthritis (Patent Document 2).

### PRIOR ART DOCUMENTS

### Patent Documents

Patent Document 1: JP 2014-9164 A
Patent Document 2: JP 2018-58781 A

### SUMMARY OF THE INVENTION

### Problems to be Solved by the Invention

As mentioned above, the extension of healthy life expectancy is an urgent issue, and to extend healthy life expectancy, it is considered useful to prevent lowering in muscle quantity and muscle strength, i.e., to improve or maintain muscle quantity and muscle strength.

### Means to Solve the Problems

In view of the above-mentioned problems, the inventors have made a diligent study and found that proteoglycans produced by a specific method can improve or maintain muscle quantity or muscle strength and can prevent obesity, or lower body fat percentage, or lower body fat quantity, or lower fat removal quantity, thereby completing the present invention.

Accordingly, the present invention provides the following.
[1] A composition comprising a proteoglycan obtained by a method for producing the proteoglycan, for improving or maintaining a muscle quantity or muscle strength, wherein
   the method for producing the proteoglycan comprises the steps of immersing a biological sample comprising the proteoglycan in an aqueous solution of a surfactant, and
   collecting the solution after the immersion.
[2] A composition comprising a proteoglycan obtained by a method for producing the proteoglycan, for preventing obesity, or lowering body fat percentage, or lowering body fat quantity, or lowering fat removal quantity, wherein
   the method for producing the proteoglycan comprises the steps of immersing a biological sample comprising the proteoglycan in an aqueous solution of a surfactant, and
   collecting the solution after the immersion.
[3] The composition described in [1] or [2], wherein the surfactant is at least one selected from the group consisting of saponin, lecithin, and sucrose fatty acid esters.
[4] The composition described in any of [1] to [3], wherein the biological sample comprising the proteoglycan is at least one selected from the group consisting of cartilage tissues, muscle fibers, and skins of fishes, mollusks, birds, and mammals.
[5] The composition described in any of [1] to [4], wherein the biological sample comprising the proteoglycan is at least one selected from the group consisting of cartilage tissues of fishes, birds, and mammals.
[6] The composition described in any of [1] to [5], which is for improving or maintaining muscle quantity or muscle strength in a healthy human subject.
[7] The composition described in any of [1] to [6], which is a cosmetic composition, a food composition, or a pharmaceutical composition.
[8] A method for improving or maintaining muscle quantity or muscle strength, comprising administering an effective amount of a proteoglycan obtained by a method for producing the proteoglycan to a subject in need thereof, wherein
   the method for producing the proteoglycan comprises the steps of immersing a biological sample comprising the proteoglycan in an aqueous solution of a surfactant, and
   collecting the solution after the immersion.
[9] A method for preventing obesity, or lowering body fat percentage, or lowering body fat quantity, or lowering fat removal quantity, comprising administering an effective amount of a proteoglycan obtained by a method for producing the proteoglycan to a subject in need thereof, wherein
   the method for producing the proteoglycan comprises the steps of
   immersing a biological sample comprising the proteoglycan in an aqueous solution of a surfactant, and
   collecting the solution after the immersion.
[10] The method described in [8] or [9], wherein the surfactant is at least one selected from the group consisting of saponin, lecithin, and sucrose fatty acid esters.
[11] The method described in [8] or [9], wherein the biological sample comprising the proteoglycan is at least one selected from the group consisting of cartilage tissues, muscle fibers, and skins of fishes, mollusks, birds, and mammals.
[12] The method described in [11], wherein the biological sample comprising the proteoglycan is at least one selected from the group consisting of cartilage tissues of fishes, birds, and mammals.
[13] The method described in [8], which is for improving or maintaining muscle quantity or muscle strength in a healthy human subject.
[14] A proteoglycan for use in improving or maintaining muscle quantity or muscle strength, wherein the proteoglycan is obtained by a method for producing the proteoglycan, comprising the steps of
   immersing a biological sample comprising the proteoglycan in an aqueous solution of a surfactant, and
   collecting the solution after the immersion.
[15] A proteoglycan for use in preventing obesity, or lowering body fat percentage, or lowering body fat quantity, or lowering fat removal quantity, wherein the proteoglycan is obtained by a method for producing the proteoglycan, comprising the steps of
   immersing a biological sample comprising the proteoglycan in an aqueous solution of a surfactant, and
   collecting the solution after the immersion.
[16] The proteoglycan described in or [15], wherein the surfactant is at least one selected from the group consisting of saponin, lecithin, and sucrose fatty acid esters.
[17] The proteoglycan described in or [15], wherein the biological sample comprising the proteoglycan is at least one selected from the group consisting of cartilage tissues, muscle fibers, and skins of fishes, mollusks, birds, and mammals.
[18] The proteoglycan described in [17], wherein the biological sample comprising the proteoglycan is at least one selected from the group consisting of cartilage tissues of fishes, birds, and mammals.
[19] The proteoglycan described in [14], which is for improving or maintaining muscle quantity or muscle strength in a healthy human subject.
[20] Use of a proteoglycan for improving or maintaining muscle quantity or muscle strength, wherein the proteoglycan is obtained by a method for producing the proteoglycan, comprising the steps of
   immersing a biological sample comprising the proteoglycan in an aqueous solution of a surfactant, and
   collecting the solution after the immersion.
[21] Use of a proteoglycan for preventing obesity, or reducing body fat percentage, or reducing body fat quantity, or reducing fat removal quantity, wherein the proteoglycan is obtained by a method for producing the proteoglycan, comprising the steps of
   immersing a biological sample comprising the proteoglycan in an aqueous solution of a surfactant, and
   collecting the solution after the immersion.

### BRIEF DESCRIPTION OF THE DRAWINGS

FIG. 1 shows the test results for muscle quantity.

FIG. 2 shows the test results of physical measurements excluding muscle quantity, and grip strength, 10-meter walk test, Time up and go Test, and blood test (baseline).

FIG. 3 shows the test results of physical measurements excluding muscle quantity, and grip strength, 10-meter walk test, Time up and go Test, and blood test (actual measurement values).

FIG. 4 shows the test results of physical measurements excluding muscle quantity, and grip strength, 10-meter walk test, Time up and go Test, and blood test (changed amounts).

### EMBODIMENTS TO CARRY OUT THE INVENTION

The proteoglycan (hereinafter also referred to as “the proteoglycan in the present invention”) to be used in the present invention is glycoproteins having extremely complex and diverse structures, and it is impossible or impractical to express their structures by structural formulas, etc., so that, in the present invention, said proteoglycan is identified by the manufacturing method.

The proteoglycan in the present invention can be produced according to the method disclosed in JP 2014-9164 A, and the method is explained below.

The method of producing the proteoglycan in the present invention comprises the steps of immersing a biological sample comprising the proteoglycan in an aqueous solution of a surfactant and collecting the solution after the immersion.

The biological sample comprising the proteoglycan includes cartilage tissues, muscle fibers, and skins of fishes, mollusks, birds, and mammals. Among these, cartilage tissues are preferred. As the cartilage tissues, any cartilage tissues of fishes, birds, and mammals, as well as waste parts of such cartilage tissues, can be used. Here, the cartilage tissue includes both cartilage alone and tissues that contain portions surrounding cartilage, such as bone, muscle fiber, skin, etc. As cartilage tissue of fish, the fins and cartilage parts of generally distributed cartilaginous fishes such as blue sharks and shortfin mako sharks as a matter of course, and the nasal cartilage tissue called ice head, which is contained in the head of salmon by an average weight of about 6%, are particularly preferred. When salmon (mostly chum salmon) caught in the coastal areas of Hokkaido are processed into various processed products, the heads are often not needed, so that, although some of the severed heads are processed into fishmeal and used, most of them are disposed of as industrial waste, and ice heads can be obtained easily, inexpensively, and stably from such waste.

In the present invention, in addition to ice head, cartilage tissue derived from fish such as cartilage tissue of ray, cartilage tissue of shark, etc., avian cartilage tissue such as cartilage tissue of chicken, and cartilage tissue of mammalian such as bovine throat cartilage and bronchial cartilage, whale cartilage, etc., can also be used. Further, it is known that proteoglycans are present in the epidermis and cartilage tissue of mollusks, such as squid and octopus, and the epidermis and cartilage, etc., of these mollusks can also be used in the present invention. In addition, in mammalian blood, a chondroitin-protein complex (bikunin), which contains little or no sulfuric acid, is present, and this chondroitin has been reported to account for almost 100% of the mucopolysaccharides in mammalian blood (Salier J. P., Rouet P., Raguenez G., Daveau M.: The inter-alpha-inhibitor family: from structure to regulation Biochem. J., 315, 1-9, 1996), and therefore, chondroitin derived from mammalian blood can also be used as a biological sample comprising the proteoglycan in the present invention. Many of the above-mentioned biological samples comprising the proteoglycans are industrial wastes and are readily available. These raw materials should be preferably pretreated by crushing them as finely as possible or defatting them before immersion in a surfactant solution, to increase their surface area and increase the amount of proteoglycans extracted.

As the surfactant, any surfactant can be used including an anionic surfactant, a cationic surfactant, a double-side surfactant, a nonionic surfactant, etc. Among these, glycerol fatty acid esters, polyglycerol fatty acid esters, propylene glycol fatty acid esters, sucrose fatty acid esters, sorbitan fatty acid esters, soy bean phospholipids (lecithin), saponin, etc., which are approved as food additives or emulsifiers for food, are preferred, and saponin, lecithin or sucrose fatty acid esters alone or mixtures of any of these are particularly preferred.

Saponin includes quillaja saponin, soy bean saponin, and tea saponin. Among these, quillaja saponin is preferred.

Lecithin includes plant lecithin, fractionated lecithin, egg yolk lecithin, enzyme-treated lecithin, and enzyme-degraded lecithin. Among these, vegetable lecithin having HLB 3 to 4 is preferred.

Sucrose fatty acid esters include sucrose fatty acid monoesters formed by the addition of one molecule of fatty acid, diesters formed by the addition of two fatty acid molecules, and triesters formed by the addition of three fatty acid molecules, to one molecule of sucrose. Theoretically, as the sucrose fatty acid esters, there can be present up to octaesters in which eight molecules of fatty acids are added, but those suitable for food use are the monoesters, diesters, triesters, and mixtures of any of these esters. Among these, in the viewpoint of emulsion stability of the emulsion, sucrose fatty acid esters having HLB 13 to 19, particularly 14 to 19, and optimally HLB 19 are preferred. If the viewpoint of reducing emulsion cost is also added, a mixture of monoesters, diesters, and triesters (hereinafter also referred to as “the sucrose fatty acid mixed esters”) is desirable, particularly a mixture of the sucrose fatty acid mixed esters having HLB 14 to 16 and sucrose fatty acid monoesters having HLB 19 are desirable. As fatty acids, saturated or unsaturated fatty acids with 10 to 22 carbon atoms are preferred, and saturated or unsaturated fatty acids with 12 to 18 carbon atoms are even more preferred.

Among the surfactants used in the present invention, sucrose fatty acid esters are preferred due to their efficiency in extracting proteoglycans and simplicity of post-treatment, and sucrose laurate, which is the sucrose fatty acid ester of lauric acid which is a saturated fatty acid having 12 carbon atoms, is particularly preferred.

The concentration of the surfactant in the aqueous surfactant solution is preferably from 0.01 to 20% by weight, particularly from 0.1 to 10% by weight, and even more preferably from 0.5 to 5% by weight. The immersion duration can be set as needed, and for example, when using a 0.1 to 2% by weight of the surfactant, for example, an aqueous solution of saponin, lecithin, or sucrose fatty acid esters, or a mixture of any of these, the immersion duration is preferably set 10 hours or longer. In addition, when a solution of 2 to 5% by weight of the surfactant, for example, saponin, lecithin, or sucrose fatty acid esters, or a mixture of any of these is used, the immersion duration is preferably set to about 12 hours. According to these conditions, proteoglycans having higher molecular weight can be efficiently collected and produced.

Immersion of the biological sample in the aqueous surfactant solution is preferably carried out at 30° C. to 100° C., more preferably at 40° C. to 80° C., and particularly preferably at 50° C. to 70° C. In particular, by setting the immersion temperature at 50° C. to 60° C., proteoglycans are hardly degraded and can be extracted as glycoprotein complexes having high molecular weight.

Immersion is carried out using preferably 1 to 50 parts by weight, more preferably 2 to 30 parts by weight, further preferably 4 to 20 parts by weight, and particularly preferably 6 to 15 parts by weight of the aqueous surfactant solution to 1 part by weight of the cartilage. It is preferable to carry out the immersion while stirring it using a mixer or stirrer, etc.

Extraction of proteoglycans from cartilage tissue can be monitored by detecting or quantifying the amount of uronic acid, for example, by the carbazole method (John et al., ANALYTICAL BIOCHEMISTRY, 1967, vol. 19, pp. 119-132), and however, uronic acid may also be detected and monitored by other known methods.

The aqueous surfactant solution that has finished immersion contains a large amount of residues after the proteoglycans have been extracted, and it is preferred to remove these by filtration, centrifugation, or any other methods.

The extract solution containing proteoglycans may be used as a product as is, and it is preferred to separate or purify the proteoglycans by an appropriate method to the purity required for the various applications of proteoglycans.

In the present invention, known purification methods can be used as purification methods of proteoglycans. The preferred purification method can include a centrifugation method. By the centrifugation method, fine solids can be easily removed as precipitation residue.

In addition, the liquid phase containing proteoglycans collected by the centrifugation method may be further filtered using filter paper or an ultrafiltration membrane separator having an appropriate fractional molecular weight, etc. The exclusion molecular weight may generally be in the range of 50,000 to 1,000,000. In this operation, if a fractional molecular weight of 500,000 or more is used, collagen can also be removed from the liquid phase, and the purity of proteoglycans can be increased. In addition, by adding water to the liquid phase containing proteoglycans to lower the viscosity of the liquid phase, the liquid can be easily passed through the membrane, and by repeating this process, it is also possible to remove a slight peculiar odor derived from the raw material.

Further, by adding the resulting concentrated solution to ethanol saturated with salt, gel-like proteoglycans can be also collected. This gel-like proteoglycan may be made into a solid using a vacuum freeze-dryer, or it may be dried using a spray dryer and made into a powdered solid.

The molecular weight of the proteoglycan in the present invention is not particularly limited as long as the purpose of the invention can be achieved, and it is preferred to have, for example, 1,000,000 or more, preferably 1,500,000 or more, and further preferably 1,800,000 or more are preferred, and although the upper limit is not particularly limited, it is preferred to have, for example, about 3,000,000 or less.

As the method for measuring the molecular weight, there may be mentioned a method using a high-performance liquid chromatography apparatus.

The composition comprising the proteoglycan in the present invention (hereinafter also referred to as the composition according to the present invention) can improve or maintain muscle quantity or muscle strength when applied to a subject.

The terms “improve or maintain muscle quantity or muscle strength” mean to improve or maintain muscle quantity or muscle strength of the subject compared to the state before application. Therefore, by the composition according to the present invention, lowering in muscle quantity or lowering in muscle strength can also be prevented.

A method for measuring muscle quantity or muscle strength is not particularly limited, and for example, the muscle quantity or muscle strength of the subject can be measured using an apparatus or device capable of measuring muscle quantity or muscle strength. As such an apparatus or device, for example, when muscle strength of the arm is to be measured, a commercially available grip strength meter can be used, and when total body muscle quantity is to be measured, a commercially available body composition analyzer can be used.

Muscles to be targeted are not particularly limited, and the muscles in the entire body can be made a subject.

For example, when the facial muscles are targeted, lowering in muscle quantity or muscle strength of the face can be prevented, and as a result, wrinkles and sagging can be prevented.

For example, when the skeletal muscles are targeted, lowering in muscle quantity or muscle strength of the muscles that operate the body can be prevented, and as a result, it is possible to prevent from becoming bedridden and locomotive syndrome, as well as to lead to prevent obesity, or to lower body fat quantity, or lower body fat quantity, or lower fat removal quantity.

The subject of application of the composition according to the present invention is not particularly limited, and includes, for example, mammals such as mice, rats, hamsters, guinea pigs, rabbits, cats, dogs, cattle, horses, sheep, monkeys, humans, etc. As the subject of the application of the composition according to the present invention, a human subject is preferred, more preferably a healthy human subject, and particularly preferably a healthy human subject who is aware of lowering in muscle quantity or muscle strength due to aging, etc.

A method for applying the composition according to the present invention to the subject is not particularly limited and can be applied orally or parenterally. Therefore, the composition according to the present invention can be prepared, for example, as cosmetic compositions, food compositions, pharmaceutical compositions, etc.

In the composition according to the present invention, if necessary, inactive ingredients known or well-known in the manufacture of cosmetic compositions, food compositions, and pharmaceutical compositions may be contained, and active ingredients other than the proteoglycan in the present invention may be contained in anticipation of enhanced action or other effects.

As the inactive ingredients to be contained in the composition according to the present invention, they are not particularly limited as long as the effects of the invention are achieved, and include, for example, carriers acceptable as cosmetics, foods, or pharmaceuticals, for example, excipients, binders, bulking agents, disintegrants, surfactants, lubricants, dispersants, buffers, preservatives, flavorings, perfumes, filming agents, carriers, and diluents.

Specific application dose and application interval of the composition according to the present invention may vary according to the weight, sex, age, condition, route of application, or other factors of the subject. Specific application doses and application intervals may be determined appropriately by those skilled in the art. However, to mention a non-limiting specific example, in general, in the case of oral administration, the daily intake of the proteoglycan in the present invention for one adult (60 kg) is, for example, 5 mg or more, preferably 10 mg or more, more preferably 20 mg or more, further preferably 30 mg or more, and preferably 1,000 mg or less, preferably 500 mg or less, further preferably 200 mg, even more preferably 100 mg or less, still further more preferably 80 mg or less, and particularly preferably 40 mg. In the present invention, it is preferable to apply such doses once to several times a day.

The period of application is not particularly limited, and repeated or continuous application is preferred, continuous application for 5 or more days is more preferred, continuous application for 12 or more weeks is still further preferred, and continuous application for 24 or more weeks is particularly preferred.

As a specific form of the composition according to the present invention, there is no specific restriction as long as the effects of the invention are achieved.

When the compositions according to the present invention are applied as cosmetic compositions or parenteral pharmaceutical compositions, they can be applied, for example, in the form of topical skin preparations. Examples of topical skin preparations include, for example, creams, lotions, emulsions, foundations, packs, foams, hard ointments, ointments, poultices, aerosols, etc. When applied as parenteral pharmaceutical compositions, they may be applied in dosage forms, for example, injections.

When the compositions according to the present invention are applied as food compositions or oral pharmaceutical compositions, they can be applied, for example, in the form of tablets, capsules, granules, powders, trochees, liquids, syrups, suspensions, etc. In addition, in the forms of food compositions, there may be also mentioned soft drinks, tea drinks, coffee drinks, fruit juice drinks, carbonated drinks, dairy drinks, jellies, wafers, cookies, bread, noodles, sausages, etc.

As a form of food compositions or pharmaceutical compositions, solid preparations such as tablets, capsules, granules, powders, trochees, etc., are preferred, and tablets and capsules are further preferred because they are easy to take, and tablets are particularly preferred.

When the composition according to the present invention is made into tablets, it is preferable to contain crystalline cellulose and a lubricant.

The content of the crystalline cellulose in the tablets is from 1 to 80% by weight, and preferably from 3 to 75% by weight. If the contained ratio of the crystalline cellulose in the tablet is too high, sticking will be likely to occur. Conversely, if the contained ratio of the crystalline cellulose content is too low, the hardness of the tablets will decrease.

The lubricant to be contained in tablets includes commonly used lubricants such as calcium stearate, silicon dioxide, calcium carboxymethylcellulose, sucrose fatty acid esters, calcium silicate, magnesium silicate, talc, and silica hydrogel, etc., and among these, calcium stearate, silicon dioxide, and calcium carboxymethylcellulose are preferred. The content of the lubricant in the tablets is 0.5 to 5% by mass and preferably 0.5 to 3% by mass. If the contained ratio of the lubricant in the tablets is too small, disorders such as sticking, capping, etc., will be likely to occur during the manufacturing process of the tablet. Conversely, if the contained ratio of the lubricant is too high, the formability of the tablet will deteriorate and sufficient tablet hardness cannot be obtained.

The tablets can be manufactured by blending raw materials simultaneously or sequentially, mixing them uniformly, and compression molding them. The compression molding method generally refers to a manufacturing method by tableting and includes a granule tableting method in which granules are granulated from the raw material powder mixture and then compression molded, and a direct tableting method in which the raw material powder mixture is directly compression molded. The tablets according to the present invention have good tableting characteristics, so that, as the manufacturing method thereof, either the granule tableting method or the direct tableting method can be used, but the direct tableting method is preferred since its process is simple and convenient. Tableting can be carried out according to usual methods using a single-shot tableting machine or a continuous tableting machine such as a rotary tableting machine, etc. Pressure for tableting can be set appropriately in consideration of the desired tablet size and hardness as described below but can be set, for example, to 2,000 to 4,000 kgf.

Size of the tablet can be a general size, for example, about 5 to 12 mm in diameter and about 100 to 400 mg in mass, but considering the ease of swallowing, it is preferable that the tablet be about 5 to 10 mm in diameter and about 200 to 300 mg in mass. The hardness of the tablet can be the hardness of a general-purpose tablet, for example, 6 kgf or more, but considering the suppression of breakage such as cracking and chipping, smoothness of the surface, and disintegration after intake, it is preferably about 6.5 to 15 kgf. The hardness of the tablet can be measured using a general-purpose tablet hardness tester (for example, Freund's tablet hardness tester).

Hereinafter the present invention will be explained in more detail by referring to Production Examples and Examples, but the present invention is not limited to these examples.

### EXAMPLES

### Production Example 1

Nasal cartilage extracted from a head of chum salmon frozen and stored at −40° C. was finely crushed and minced using an electric meat chopper. The minced nasal cartilage was defatted and dehydrated. The nasal cartilage after the treatment was ventilated or vacuum-dried and 24 g of the resulting material was used as a starting material. In a 5-liter extraction vessel was charged 2,970 g of distilled water previously cooled to 5° C., and 30 g of the solid-state sucrose fatty acid esters used in Production Example 1 were added thereto to prepare a total of 3,000 g (1% by weight) of an aqueous sucrose fatty acid ester solution. In this extraction vessel was charged 24 g of the starting material and immersed for 12 hours while stirring using a stirrer.

After the completion of the immersion, the contents were transferred to another vessel set with a 1 mm square strainer made of stainless steel, the nasal cartilage was removed, and the extract solution containing proteoglycans was collected.

The extract solution was centrifuged in a Hitachi himac CF7D2 type centrifuge at 3,000 rpm for 20 minutes to remove solid components and oil and fat components, and the liquid phase containing proteoglycans was collected.

Further, this liquid phase was filtered using a filter paper (available from Advantech), and after adding distilled water 6 times the volume of the filtrate, fractionation and concentration were carried out simultaneously using BIOMAX 100K POLYETHERSULFONE (fractional molecular weight 100,000) available from Nihon Millipore K.K.

A portion of the resulting concentrated solution was taken and the weight of the solid contents in the solution was measured. For the measurement, the solution was dried in a drying oven (YAMATO DX401) at 105° C. for 16 hours to completely evaporate the water content, and the remaining solid content was precisely measured with a digital weighing instrument (GF-400 manufactured by A&D). As a result, from 24 g of the starting material, 5.5 g of dried solid content was obtained, which is equivalent to 22.21% of the starting material in terms of conversion value.

In addition, the amount of collagen in the concentrated solution was determined by measuring the amount of amino acids using an automatic amino acid analyzer (L-8500 Amino Acid Analyzer, manufactured by Hitachi, Ltd.), and the amount of uronic acid was determined by the Galambos method to calculate the amount of proteoglycans. Further, the molecular weight of the proteoglycan was determined using a high-performance liquid chromatography device (column TSK-GEL G4000PWXL, manufactured by Shimadzu Corporation).

From the results of these analyses, it was found that 14.8% of protein, 18.4% of ash, 64.8% of carbohydrate, and 0% of lipid were present in the solid content. Also, the molecular weight of the proteoglycan was approximately 1,300,000.

### Production Example 2

Nasal cartilage extracted from a head of chum salmon frozen and stored at −40° C. was finely crushed and minced using an electric meat chopper. The minced nasal cartilage was defatted and dehydrated. The nasal cartilage after the treatment was ventilated or vacuum-dried and 17.9 g of the resulting material was used as a starting material. In a 5-liter extraction vessel was charged 2,495 g of distilled water previously cooled to 5° C., and 5 g of the solid-state quillaja saponin was added thereto to prepare a total of 2,500 g (0.2% by weight) of aqueous saponin solution. In this extraction vessel was charged 17.9 g of the starting material and immersed for 12 hours while stirring using a stirrer.

After the completion of the immersion, the contents were transferred to another vessel set with a 1 mm square strainer made of stainless steel, the nasal cartilage was removed, and the extract solution containing proteoglycans was collected.

The extract solution was centrifuged in a Hitachi himac CF7D2 type centrifuge at 3,000 rpm for 20 minutes to remove solid components and oil and fat components, and the liquid phase containing proteoglycans was collected.

Further, this liquid phase was filtered using a filter paper (available from Advantech), and after adding distilled water 6 times the volume of the filtrate, fractionation and concentration were carried out simultaneously using BIOMAX 100K POLYETHERSULFONE (fractional molecular weight 100,000) available from Nihon Millipore K.K.

A portion of the resulting concentrated solution was taken and the weight of the solid contents in the solution was measured. For the measurement, the solution was dried in a drying oven (YAMATO DX401) at 105° C. for 16 hours to completely evaporate the water content, and the remaining solid content was precisely measured with a digital weighing instrument (GF-400 manufactured by A&D). As a result, from 17.9 g of the starting material, 3.29 g of dried solid content was obtained, which is equivalent to 18.41% of the starting material in terms of conversion value.

In addition, the amount of collagen in the concentrated solution was determined by measuring the amount of amino acids using an automatic amino acid analyzer (L-8500 Amino Acid Analyzer, manufactured by Hitachi, Ltd.), and the amount of uronic acid was determined by the Galambos method to calculate the amount of proteoglycans. Further, the molecular weight of the proteoglycan was determined using a high-performance liquid chromatography device (column TSK-GEL G4000PWXL, manufactured by Shimadzu Corporation).

From the results of these analyses, it was found that 17% of protein, 22.4% of ash, 60.6% of carbohydrate, and 0% of lipid were present in the solid content. Also, the molecular weight of the proteoglycan was approximately 1,200,000. [Production Example 3]

200 g of nasal cartilage extracted from a shortfin mako shark frozen at −40° C. and finely crushed and minced using an electric meat chopper was prepared and used as a starting material. In a 5-liter extraction vessel was charged 2,376 g of distilled water previously cooled to 0° C., and 24 g of solid-state sucrose fatty acid esters (C12: HLB 14 to 18) were added thereto to prepare a total of 2,400 g (1% by weight) of an aqueous sucrose fatty acid esters solution. In this extraction vessel was charged 200 g of the starting material and immersed for 12 hours while stirring using a stirrer.

After the completion of the immersion, the contents were transferred to another vessel set with a 1 mm square strainer made of stainless steel, and the extract solution containing proteoglycans was collected.

The extract solution was centrifuged using an IWAKI CFS-400 centrifuge at 1,500×g for 30 minutes to remove solid components and oil and fat components, and the liquid phase containing proteoglycans was collected.

Further, this liquid phase was filtered using a filter paper (available from Advantech), and after adding distilled water 6 times the volume of the filtrate, fractionation and concentration were carried out simultaneously using a PREP/SCALE TFF membrane (fractional molecular weight 100,000) available from Nihon Millipore K.K.

A portion of the resulting concentrated solution was taken and the weight of the solids in the solution was measured. For the measurement, the solution was dried in a drying oven (YAMATO DX401) at 105° C. for 16 hours to completely evaporate the water content, and the remaining solid content was precisely measured with a digital weighing instrument (GF-400 manufactured by A&D). As a result, from 200 g of the starting material, 5.5 g of dried solid content was obtained, which is equivalent to 2.75% of the starting material in terms of conversion value.

In addition, the amount of collagen in the concentrated solution was determined by measuring the amount of amino acids using an automatic amino acid analyzer (L-8500 Amino Acid Analyzer, manufactured by Hitachi, Ltd.), and the amount of uronic acid was determined by the carbazole method to calculate the amount of proteoglycans.

Further, the molecular weight of the proteoglycan was determined using a high-performance liquid chromatography device (column Asahipak, manufactured by Shimadzu Corporation).

From the results of these analyses, it was found that 35% of protein, 21.5% of ash, 42.9% of carbohydrate, and 0.6% of lipid were present in the solid content. According to Patent Document 1, the weight ratio of the core protein of a proteoglycan is disclosed as about 7%, and therefore, the purity of the proteoglycan in the present invention is estimated to be about 46%, calculated from the fact that the carbohydrates are about 42.9%. Also, the molecular weight of the proteoglycan was approximately 2,000,000.

In the operation shown in the above-mentioned Production Example 3, changes with a lapse of time in the amount of proteoglycans collected (amount of uronic acid) when the concentration of sucrose fatty acid ester solution was changed to 0.1%, 5%, and 10% and immersed for 24 hours and extracted was examined, almost similar results were obtained.

In addition, in the operation shown in the above-mentioned Production Example 1, changes with a lapse of time in the amount of proteoglycans collected (amount of uronic acid) when the sucrose fatty acid ester was changed to 0.666 N acetic acid and immersed for 24 hours and extracted was examined, and the results were inferior to those in Production Example 3.

### Examples

The efficacy of the composition according to the present invention was tested by a placebo-controlled randomized double-blind parallel-group comparison method.

### 1. Formulation of the Test Composition

In the following Examples, the proteoglycan produced in Production Example 1 (hereinafter also referred to as “the proteoglycan complex”) was used, and the following formulation of the test composition was used.

TABLE 1
Formulation of test composition (per one tablet)

| | | | Control composition | |
|---|---|---|---|---|
| | Test food | | (placebo) | |
| | | Ratio | | Ratio |
| | Formulation | (% by | Formulation | (% by |
| Name of raw material | (mg) | weight) | (mg) | weight) |
| Proteoglycan complex | 40 | 20 | — | — |
| Crystalline cellulose | 150 | 75 | 190 | 95 |
| Calcium stearate | 4 | 2 | 4 | 2 |
| Fine particulate silicon | 3 | 1.5 | 3 | 1.5 |
| dioxide | | | | |
| Carboxymethyl cellulose | 3 | 1.5 | 3 | 1.5 |
| Total | 200 | 100 | 200 | 100 |

Each ingredient was mixed in the proportions that would result in one tablet having the formulation described above, and the resulting mixture was directly pressed to produce tablets.

2. Test period

24 weeks

### 3. Administration Method

One tablet was taken to a subject once a day with water or hot water after supper.

### 4. Subjects

The following persons were selected as subjects.
(1) Enrollment criteria
   i. Japanese
   ii. Adult male and female
   iii. Healthy subjects
   iv. Persons who are experiencing weakening in muscle strength.
(2) Exclusion criteria
   i. Persons under treatment for or with a history of malignancy, heart failure, or myocardial infarction
   ii. Persons with pacemakers or implantable cardioverter-defibrillators
   iii. Persons under treatment for the following chronic diseases:
      Arrhythmia, hepatic disorder, renal disorder, cerebrovascular disorder, rheumatism, diabetes mellitus, dyslipidemia, hypertension, and other chronic diseases
   iv. Persons diagnosed with sarcopenia
   v. Persons who regularly take special health food, food with functional labeling, or other food/beverage with possible functionality
   vi. Persons who regularly use pharmaceuticals (including herbal medicines) and/or supplements
   vii. Persons who have allergies (to pharmaceuticals or test food-related foods)
   viii. Persons who are pregnant, lactating, or intend to become pregnant during the study period
   ix. Participated in another clinical trial in the 3 months before the date of consent, or intends to do so during the study period
   x. Other persons who are deemed inappropriate as a subject for the study by the study directional doctor
(3) Selection criteria
   i. Persons who are judged by the study directional doctor to be fit to participate in the study
   ii. Those who meet two or more of the following criteria will be excluded.
      a. Persons whose grip strength is less than 28 kg in males, and less than 18 kg in females
      b. Persons whose normal walking speed is less than 1 m/s
      c. Persons whose skeletal muscle quantity is less than 7.0 kg/m² in males and 5.7 kg/m² in females
   iii. Persons with relatively low values of muscle quantity
5. Evaluation items

The following items were evaluated.
(1) Grip strength
   i. Content of implementation items: Grip strength will be examined at the trial medical institution using a grip strength meter.
   ii. Investigation item: Grip strength
   iii. Evaluation method: Measurements will be taken at screening and test before intake, test after 12 weeks of intake, and test after 24 weeks of intake.
(2) 10-meter walk test
   i. Content of implementation items: A 10-meter walking test is carried out to evaluate walking ability.
   ii. Investigation items: Walking time, number of steps, stride length, and walking rate (number of steps per unit time).
   iii. Evaluation method: Measurements will be taken at screening and test before intake, test after 12 weeks of intake, and test after 24 weeks of intake.
(3) Time up and go Test
   i. Content of implementation items: Time up and go Test is carried out to evaluate walking ability.
   ii. Investigation items: Normal walking speed, maximum walking speed
   iii. Evaluation method: Measurements will be taken at screening and test before intake, test after 12 weeks of intake, and test after 24 weeks of intake.

### 6. Outcomes

(1) Efficacy evaluation items
   i. Primary outcome
      a. Actual measurement value of muscle quantity after 24 weeks of intake
   ii. Secondary outcomes
      a. Actual measurement value of muscle quantity after 12 weeks of intake
      b. Changed amount in muscle quantity after 12 weeks of intake, and after 24 weeks of intake from screening and test before intake
      c. Actual measurement values after 12 weeks of intake and after 24 weeks of intake and changed amounts from screening and test before intake in grip strength, walking time, number of steps, stride length, walking rate, normal walking speed, maximal walking speed, body fat percentage, body fat quantity, fat removal quantity, body weight, high-sensitivity CRP, Total PINP, and BAP

### 7. Test Results

### 1. Test Participants

The present test included healthy Japanese adult men and women who were experiencing weakness in muscle strength. Of the 114 participants who agreed to participate in the test, 56 participants who met the eligibility criteria were included in the present test, and 28 participants were assigned to the test food group and 28 participants to the placebo group.

One test participant (one in the test food group) did not return to the hospital after 24 weeks of intake.

In Per protocol set (PPS), one participant (one in the test food group) with no post-assignment data and 7 participants (4 in the placebo group and 3 in the test food group) with less than 80% intake of the test product were excluded. In Safety analysis population (SAF), one participant (one in the test food group) who did not have any safety endpoints measured after allocation was excluded. Thus, the number of cases in each analysis data set was 48 participants for the PPS (24 participants in the placebo group and 24 participants in the test food group) and 55 participants for the SAF (28 participants in the placebo group and 27 participants in the test food group). The background of the test participants is shown in the following Table.

TABLE 2
Background of participants (PPS)
Background

| | PPS | | |
|---|---|---|---|
| | Placebo group | Test food group | Significant |
| Test items | (n = 24) | (n = 24) | probability |
| Sexuality (male) | 2 (8.3%) | 2 (8.3%) | 1.000 |
| Sexuality (female) | 22 (91.7%) | 22 (91.7%) | |
| Age (years) | 51.0 ± 10.0 | 49.0 ± 11.2 | 0.509 |
| Body height (cm) | 160.4 ± 6.3 | 159.3 ± 6.0 | 0.545 |
| Body weight (kg) | 52.3 ± 8.7 | 52.6 ± 5.6 | 0.879 |
| BMI (kg/m²) | 20.3 ± 2.8 | 20.8 ± 2.7 | 0.531 |
| Systolic blood | 115.0 ± 11.9 | 116.4 ± 12.5 | 0.698 |
| pressure (mmHg) | | | |
| Diastolic blood | 74.9 ± 8.3 | 73.6 ± 8.9 | 0.607 |
| pressure (mmHg) | | | |
| Pulse rate (bpm) | 73.3 ± 12.5 | 70.3 ± 10.1 | 0.366 |

Background of participants (SAF)
Background

| | SAF | | |
|---|---|---|---|
| | Placebo group | Test food group | Significant |
| Test items | (n = 28) | (n = 27) | probability |
| Sexuality (male) | 2 (7.1%) | 2 (7.4%) | 1.000 |
| Sexuality (female) | 26 (92.9%) | 25 (92.6%) | |
| Age (years) | 49.4 ± 11 | 49.8 ± 10.9 | 0.887 |
| Body height (cm) | 160.7 ± 6 | 159.3 ± 5.7 | 0.383 |
| Body weight (kg) | 52.4 ± 8.4 | 52.3 ± 5.6 | 0.938 |
| BMI (kg/m²) | 20.3 ± 2.8 | 20.7 ± 2.6 | 0.613 |
| Systolic blood | 113.9 ± 11.7 | 115.5 ± 12.1 | 0.615 |
| pressure (mmHg) | | | |
| Diastolic blood | 74.5 ± 7.9 | 73.2 ± 8.6 | 0.557 |
| pressure (mmHg) | | | |
| Pulse rate (bpm) | 72.6 ± 12.8 | 70.5 ± 10.4 | 0.511 |

Sexuality is indicated by the corresponding n number and percentage occupied in the group, and was compared between groups using the χ-square test. Others are presented as mean value (Mean) and standard deviation (SD) and were compared between groups using Student's t-test.

### 2. Primary Outcome

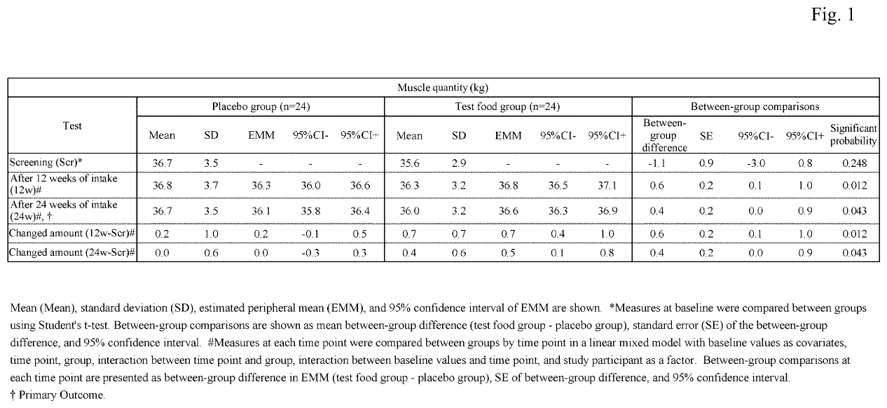

The mean value (Mean) and standard deviation (SD) of muscle quantity, 5 estimated peripheral mean (EMM) and its 95% confidence interval (95% CI−, 95% CI+), between-group differences in EMM and its 95% CI−, 95% CI+, and statistical analysis results are shown in FIG. 1.

The measured muscle quantity at 24 weeks after intake (24w) was significantly higher in the test food group compared to the placebo group (P=0.043).

### 3. Secondary Outcomes

### 3-1. Muscle Quantity (Excluding Primary Outcome)

Mean and SD of muscle quantity, EMM and its 95% CI−, 95% CI+, between-group difference in EMM {between-group difference in Mean at screening and test before intake (Scr)} and its 95% CI−, 95% CI+, and statistical analysis results are shown in FIG. 1.

### (1) Actual Measurement Values

Of the time points at which significant differences were observed, the time point at which the test food group was higher than the placebo group was the 12 weeks after intake test (12w) (P=0.012).

### (2) Amount of Change

Of the time points at which significant differences were observed, the time points at which the test product group was higher than the placebo group were 12w (P=0.012) and 24w (P=0.043).

### 3-2. Physical Measurements Excluding Muscle Quantity, and Grip Strength, 10-Meter Walk Test, Time Up and go Test, and Blood Test

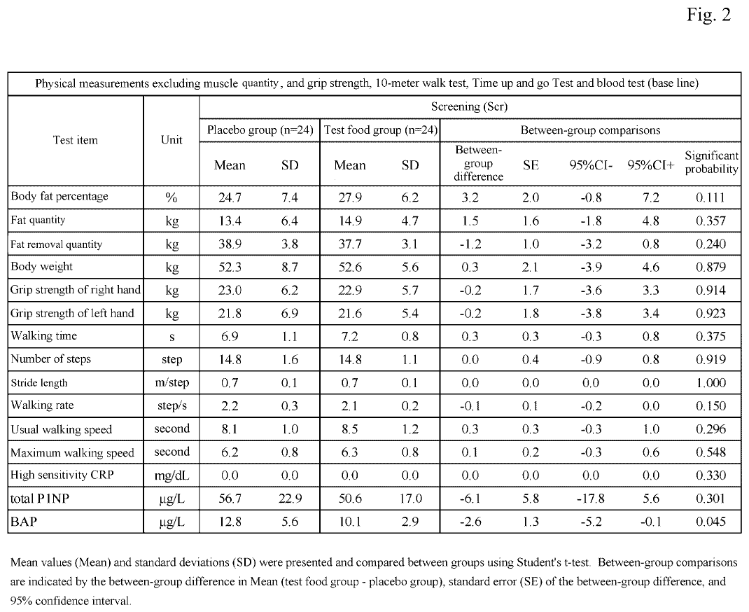

Mean and SD, between-group differences and 95% CI−, 95% CI+, and statistical analysis results for physical measurements excluding muscle quantity, and grip strength, 10-meter walk test, Time up and go Test, and blood test are shown in FIGS. 2 to 4.

### (1) Scr

Among the items for which significant differences were observed, the item for which the test food group showed lower values than the placebo group was BAP (P=0.045).

### (2) Actual Measurement Values

a. 12w

Among the items for which significant differences were observed, the items for which the test food group showed lower value than the placebo group were body fat percentage (P=0.019) and fat quantity (P=0.031), and the item for which high values were observed was fat removal quantity (P=0.014).

b. 24w.

Among the items for which significant differences were observed, the items for which the test food group showed lower value than in the placebo group were body fat percentage (P=0.008) and fat quantity, (P=0.005), and the item for which high values were observed was fat removal quantity (P=0.042).

### (3) Changed Amount

a. 12w-Scr

Among the items for which significant differences were observed, the items for which the test food group showed lower value than the placebo group were body fat percentage (P=0.019) and fat quantity (P=0.031), and the item for which high values were observed was fat removal quantity (P=0.014).

b. 24w-Scr

Among the items for which significant differences were observed, the items for which the test food group showed lower value than in the placebo group were body fat percentage (P=0.008) and fat quantity (P=0.005), and the item for which high values were observed was fat removal quantity (P=0.042).

### 8. Consideration

Muscle quantity after 24 weeks of intake set to the primary outcome showed significantly higher values in the test food group than in the placebo group (FIG. 1), with an estimated peripheral mean (EMM) and 95% confidence interval of 36.6 kg (36.3 to 36.9) for the test food group and 36.1 kg (35.8 to 36.4) for the placebo group, and the difference between the groups was 0.4 kg (0.0 to 0.9). In addition, the changed amount from the screening and test before intake after 24 weeks of intake showed significantly higher values in the test food group than in the placebo group, and the EMM and its 95% confidence interval were 0.5 kg (0.1 to 0.8) for the test food group and 0.0 kg (−0.3 to 0.3) for the placebo group, and the difference between groups was 0.4 kg (0.0 to 0.9) (FIG. 1). This test was conducted from winter to summer for screening and test before intake and test after 24 weeks of intake, and it has been reported that fat removal quantity (weight of muscles, organs, etc. excluding fat from body weight) changes by 0.3 to 0.4 kg from winter to summer (Takaharu Ikeuchi, Taketoshi Morimoto, and Hiroyasu Nishikawa, Seasonal change in body composition assessed by bioelectrical impedance method: comparison of elderly and adolescents, Japanese Journal of Biometeorology, 1994: 31 (2): 69-73). Further, according to the report by Oka et al. (Takuya Oka, Genkai-Kato M., Variation in body weight and body composition with special attention to body in humans and factors that cause changes in body fat percentage, Kuroshio Science, 2012: 5 (2): 161-7), who observed changes in body composition over 7 years in the same male, a variation range in skeletal muscle percentage over 7 years is 0.7% and when applied to the muscle quantity in this study, it is just under 0.4 kg. From the above, a change of 0.4 kg or more in muscle quantity is considered clinically significant. In addition, the change in muscle quantity of 0.4 kg or more was confirmed after 12 weeks of intake, and both the actual measurement values and the changed amounts from the screening and test before intake were higher in the test food group than in the placebo group (FIG. 1). And the EMM and 95% confidence interval for the difference between groups were 0.6 kg (0.1 to 1.0), suggesting that the intake of the test food contributed to the clinically significant increase in muscle quantity after 12 weeks of intake.

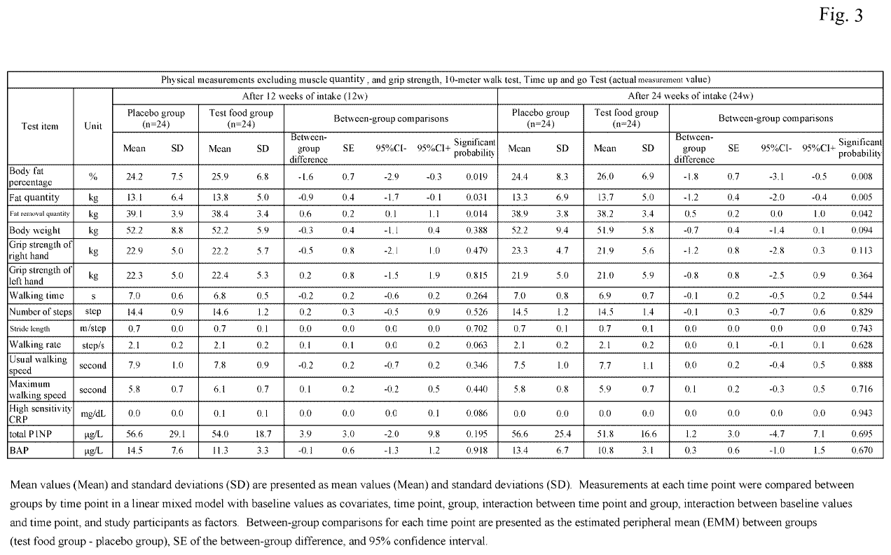

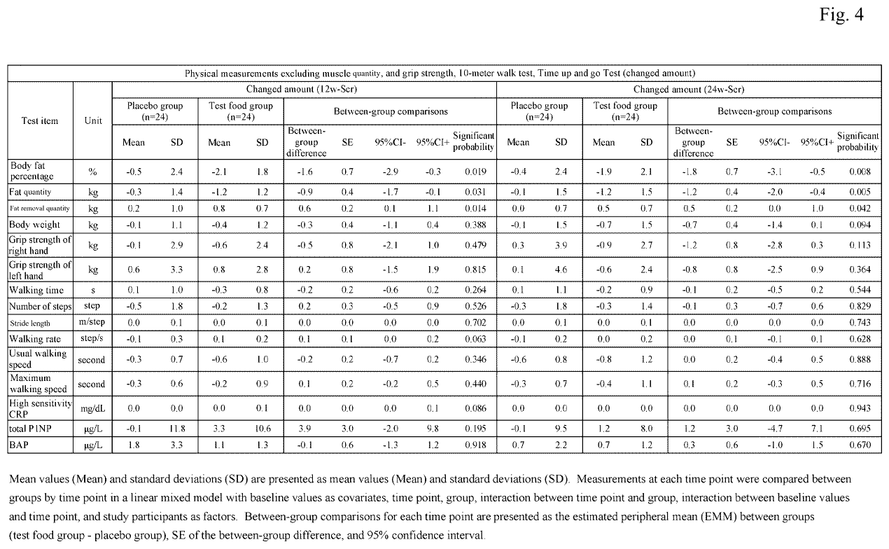

After 12 weeks of intake and after 24 weeks of intake, significant differences between groups were admitted in body fat percentage, fat quantity, and fat removal quantity, in addition to muscle quantity (FIGS. 3 and 4). A measurement method of the body composition in this study was a bioelectrical impedance analysis (BIA method) using a body composition analyzer MC-780A-N33). The fat removal quantity is determined from the body height and the resistance (impedance) of the body, and the majority of the fat removal quantity is muscle tissue, so that the muscle quantity measured by the BIA method is estimated from the fat removal quantity. Therefore, if the amount of skeletal muscle in the body increases, fat removal quantity and muscle quantity will increase simultaneously. On the other hand, fat quantity is calculated by subtracting fat removal quantity from body weight, and there was no effect of the intake of the test food on body weight (FIGS. 3 and 4), so that an increase in fat removal quantity will inevitably result in a decrease in fat quantity based on the measurement principle of the BIA method. From the above, it can be considered that the increase in skeletal muscle quantity due to the intake of the test food increased fat removal quantity, which inevitably decreased fat quantity, resulting in the decrease in body fat percentage, and the significant difference between the groups was observed.

### 9. Conclusion

From the results of these Examples, it was confirmed that the composition according to the present invention is effective in improving or maintaining muscle quantity or muscle strength, and in preventing obesity, lowering body fat percentage, lowering body fat quantity, or lowering fat removal quantity.

### INDUSTRIAL APPLICABILITY

According to the present invention, it is possible to improve or maintain muscle quantity or muscle strength, and thereby prevent obesity, lower body fat percentage, lower body fat quantity, or lower fat removal quantity. In addition, the proteoglycan used in the present invention is of biological origin and has low toxicity. Further, since the proteoglycan used in the present invention can be produced from raw materials that would normally be discarded, the present invention can contribute to the effective use of resources and can also reduce the rise in medical costs, etc.

This application is based on Patent Application No. 2022-124661 filed in Japan, all the contents of which are included herein.

1-7. (canceled)

8. A method for improving or maintaining muscle quantity or muscle strength, comprising
administering an effective amount of a proteoglycan obtained by a method for producing the proteoglycan to a subject in need thereof, wherein
the method for producing the proteoglycan comprises the steps of immersing a biological sample comprising the proteoglycan in an aqueous solution of a surfactant, and
collecting the solution after the immersion.

9. A method for preventing obesity, or lowering body fat percentage, or lowering body fat quantity, or lowering fat removal quantity, comprising administering an effective amount of a proteoglycan obtained by a method for producing the proteoglycan to a subject in need thereof,
wherein
the method for producing the proteoglycan comprises the steps of immersing a biological sample comprising the proteoglycan in an aqueous solution of a surfactant, and
collecting the solution after the immersion.

10. The method according to claim 8, wherein the surfactant is at least one selected from the group consisting of saponin, lecithin, and sucrose fatty acid esters.

11. The method according to claim 8, wherein the biological sample comprising the proteoglycan is at least one selected from the group consisting of cartilage tissues, muscle fibers, and skins of fishes, mollusks, birds, and mammals.

12. The method according to claim 11, wherein the biological sample comprising the proteoglycan is at least one selected from the group consisting of cartilage tissues of fishes, birds, and mammals.

13. The method according to claim 8, which is for improving or maintaining muscle quantity or muscle strength in a healthy human subject.

14-21. (canceled)

22. The method according to claim 9, wherein the surfactant is at least one selected from the group consisting of saponin, lecithin, and sucrose fatty acid esters.

23. The method according to claim 9, wherein the biological sample comprising the proteoglycan is at least one selected from the group consisting of cartilage tissues, muscle fibers, and skins of fishes, mollusks, birds, and mammals.

24. The method according to claim 23, wherein the biological sample comprising the proteoglycan is at least one selected from the group consisting of cartilage tissues of fishes, birds, and mammals.
